(19) United States
(12) Patent Application Publication
(10) Pub. No.: US 2009/0281586 A1
(43) Pub. Date: Nov. 12, 2009

(54) IMPLANTABLE PULSE GENERATOR EMU FILTERED FEEDTHRU

(75) Inventor: Wisit Lim, Palmdale, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/117,090

(22) Filed: May 8, 2008

(51) Int. Cl. A61N 1/00

(52) U.S. Cl. 607/5

(57) ABSTRACT

Disclosed herein is an implantable pulse generator. The implantable pulse generator may include a header, a can and a feedthru. The header may include a lead connector block electrically coupled to a first conductor. The can may be coupled to the header and include a wall and an electronic component electrically coupled to a second conductor and housed within the wall. The feedthru may be mounted in the wall and include a header side with a first electrically conductive tab and a can side with a second electrically conductive tab electrically coupled to the first tab. The first tab is electrically coupled to the first conductor and the second tab is electrically coupled to the second conductor.

### FIELD OF THE INVENTION

The present invention relates to medical apparatus and methods. More specifically, the present invention relates to feedthrus for implantable pulse generators and methods of manufacturing such feedthrus.

### BACKGROUND OF THE INVENTION

Implantable pulse generators, such as pacemakers, defibrillators or implantable cardioverter defibrillators (“ICD”), are used to provide electrotherapy to cardiac tissue via implantable medical leads. An implantable pulse generator feedthru is used for an electrical pathway extending between the electrically conductive lead securing components of a header of the pulse generator and the electrical components, such as an output flex, hybrid, etc., hermetically sealed in the housing or can of the pulse generator.

Feedthrus are mounted in the wall of the housing or can and include feedthru wires extending through the feedthrus. Feedthrus provide insulated passageways for feedthru wires, such as platinum iridium (Pt/Ir) wires, through the wall of the can. The header ends of the feedthru wires are electrically connected to connector blocks that mechanically and electrically couple with connector ends of implantable medical leads, and the can ends of the feedthru wires are electrically connected to the electrical components housed in the can of the pulse generator.

There are a number of disadvantages associated with current feedthru designs. First, current feedthrus have feedthru wires that extend through the feedthru, which is an expensive configuration due to the labor involved with manufacturing and the substantial lengths of Pt/Ir wire needed for such feedthru wires. Second, current feedthrus employ discoidal filter assemblies for filtering out unwanted signals, such as those associated with electromagnetic interference (“EMI”). Discoidal filter assemblies have high associated material and manufacturing costs.

There is a need in the art for a feedthru that has reduced material and manufacturing costs. Also, there is a need in the art for a method of manufacturing such a feedthru.

### BRIEF SUMMARY OF THE INVENTION

Disclosed herein is an implantable pulse generator. In one embodiment, the implantable pulse generator includes a header, a can and a feedthru. The header may include a lead connector block electrically coupled to a first conductor. The can may be coupled to the header and include a wall and an electronic component electrically coupled to a second conductor and housed within the wall. The feedthru may be mounted in the wall and include a header side with a first electrically conductive tab and a can side with a second electrically conductive tab electrically coupled to the first tab. The first tab is electrically coupled to the first conductor and the second tab is electrically coupled to the second conductor. In one embodiment, a chip capacitor may be located on a can side of the feedthru.

Disclosed herein is an implantable pulse generator feedthru. In one embodiment, the feedthru includes: an electrically insulating body including a header side and a can side; a ground circuit at least a portion of which is on the body; and a power circuit including a first tab on one of the sides. In one embodiment, the feedthru may further include a chip capacitor coupled to the body and including a power side electrically coupled to the power circuit and a ground side electrically coupled to the ground circuit.

Disclosed herein is an implantable pulse generator feedthru. In one embodiment, the feedthru includes: an electrically insulating body including a header side and a can side; a ground side conductive path operably coupled to the body; and a power side conductive path extending through the body, wherein the conductive path is not a feedthru wire. In one embodiment, the feedthru further includes a chip capacitor coupled to the body and including a power side electrically coupled to the power side conductive path and a ground side electrically coupled to the ground side conductive path

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an isometric view of an implantable pulse generator employing a feedthru according to the present disclosure.

FIG. 2A is a top isometric view of the feedthru of FIG. 1.

FIG. 2B is a top plan view of an alternative embodiment of the feedthru of FIG. 1.

FIG. 3A is a bottom isometric view of the feedthru of FIG. 1.

FIG. 3B is a bottom plan view of an alternative embodiment of the feedthru of FIG. 1.

FIG. 4 is a side view of the feedthru taken from the direction of arrow “A” of FIG. 3A.

FIG. 5A is a longitudinal cross-sectional elevation of the feedthru as taken along section line 5A-5A of FIG. 4.

FIG. 5B is a longitudinal cross-sectional elevation of an alternative embodiment of the feedthru as taken along section line 5B-5B of FIG. 2B, wherein the chip capacitors are not shown for clarity purposes.

FIGS. 6A-6F are cross-sectional views of alternative tab/via configurations as if viewed in region A of FIG. 5A.

FIG. 7A is a cross-sectional view of a low-relief generally flush tab configuration as if viewed in region A of FIG. 5A.

FIG. 7B is an isometric view of the tab configuration of FIG. 7A as if viewed in region B of FIG. 2A.

FIG. 7C is a cross-sectional view of a low-relief bump tab configuration as if viewed in region A of FIG. 5A.

FIG. 7D is an isometric view of the tab configuration of FIG. 7C as if viewed in region B of FIG. 2A.

FIG. 7E is a cross-sectional view of post-type tab having a spherical configuration as if viewed in region A of FIG. 5A.

FIG. 7F is an isometric view of the tab configuration of FIG. 7E as if viewed in region B of FIG. 2A.

FIG. 7G is a cross-sectional view of a post-type tab having a cylindrical configuration as if viewed in region A of FIG. 5A.

FIG. 7H is an isometric view of the tab configuration of FIG. 7G as if viewed in region B of FIG. 2A.

### DETAILED DESCRIPTION

The present disclosure describes a feedthru 55 of an implantable pulse generator 5 such as a defibrillator, a pacemaker or an ICD. The feedthru 55 disclosed herein includes tabs 70 for electrical communication between the components of the header 10 (e.g., the connector blocks 20) and the electrical components 17 (e.g., output flex, hybrid, etc.) housed within the can 15. The feedthru 55 provides an electrically insulated passageway for electrical communication via the tabs 70 through the can wall 65.

Generally, the tabs 70 and the components 20, 17 of the header 10 and the can 15 are in electrical communication via conductors 60, 62 such as round wire, flat ribbon wire, flex cable, etc. The feedthru 55 reduces manufacturing and material costs because it does not employ feedthru wires, which are typically made of expensive Pt/Ir. The feedthru 55 further reduces material and design costs by utilizing an off-the-shelf chip capacitor 90 as an EMI filter element, the chip capacitor 90 being less expensive than a discoidal capacitor with respect to material and manufacturing costs. Due in part to its lack of feedthru wires, the feedthru 55 is generally compact and low profile and can therefore be installed in the inclined portion 80 and/or the flat portion 85 of the can 15 or any other part of the can 15 including the vertical side walls.

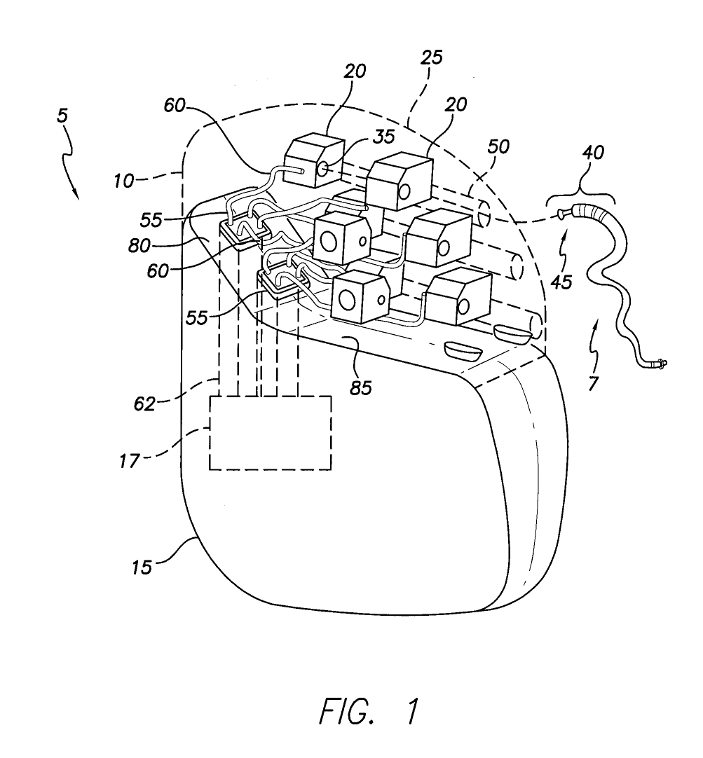

For a general discussion of an implantable pulse generator 5 that utilizes the feedthru 55 disclosed herein, reference is first made to FIG. 1, which is an isometric view of such an implantable pulse generator 5. As indicated in FIG. 1, the pulse generator 5 includes a header 10 and a can or housing 15. The header 10 includes connector blocks 20 and a molded portion 25 (shown in phantom) that encloses the blocks 20. Each block 20 includes an opening 35 configured to receive therein and mate with a connector end 40 of a lead proximal end 45, thereby forming an electrical connection between the connector block 20 and the lead connector end 40 and mechanically securing the proximal end 45 of the lead 7 to the header 10 of the pulse generator 5.

The header molded portion 25 (shown in phantom) may be formed of a polymer material. Passages 50 (shown in phantom) extend from the exterior of the molded portion 25 to the openings 35 in the blocks 20, providing a pathway for the lead distal ends 40 to pass through the molded portion 25 and enter the openings 35.

The can 15 includes feedthrus 55 mounted in the wall of the can 15. Conductors 60 (e.g., round wires, flat ribbon wires, flex cables or etc.) extend from the header sides of the feedthrus 55 to respective connector blocks 20. The can 15 provides a hermetically sealed enclosure for the pulse generator's electronic components 17 (e.g., output flex, hybrid, or various other electronic components) housed within the can 15. Conductors 62 (e.g., round wires, flat ribbon wires, flex cables or etc.) extend from the can sides of the feedthrus 55 to the electronic components 17. Typically, the wall of the can 15 is made of titanium or another biocompatible metal.

As shown in FIG. 1, in one embodiment, the feedthrus 55 are mounted in an inclined portion 80 of the can 15. In other embodiments, the feedthrus 55 may be mounted in a flat portion 85 of the pulse generator 5, or the feedthrus 55 may be mounted in both the inclined and flat portions 80, 85 of the can 15. In yet other embodiments, the feedthrus 55 may be mounted on the vertical side walls of the can 15.

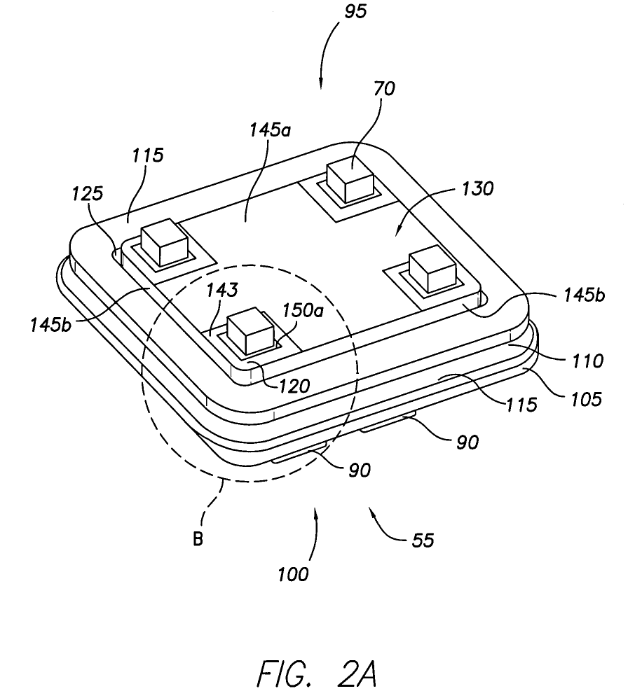

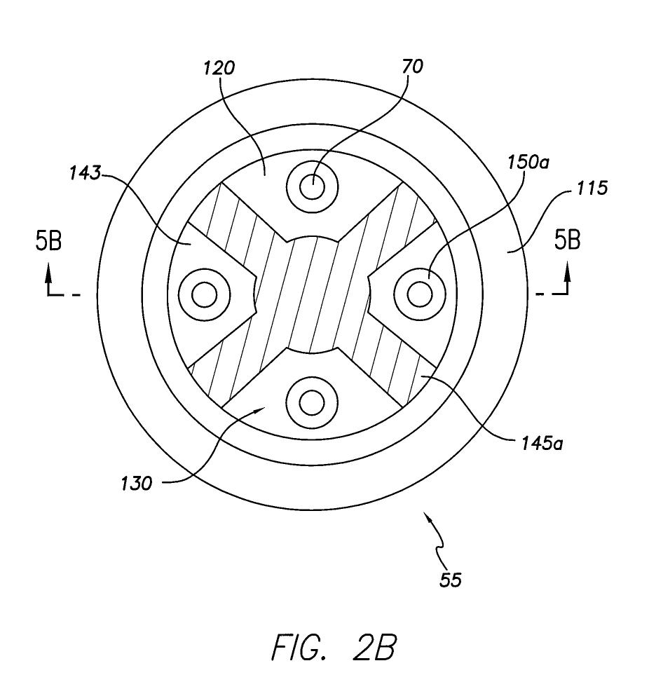

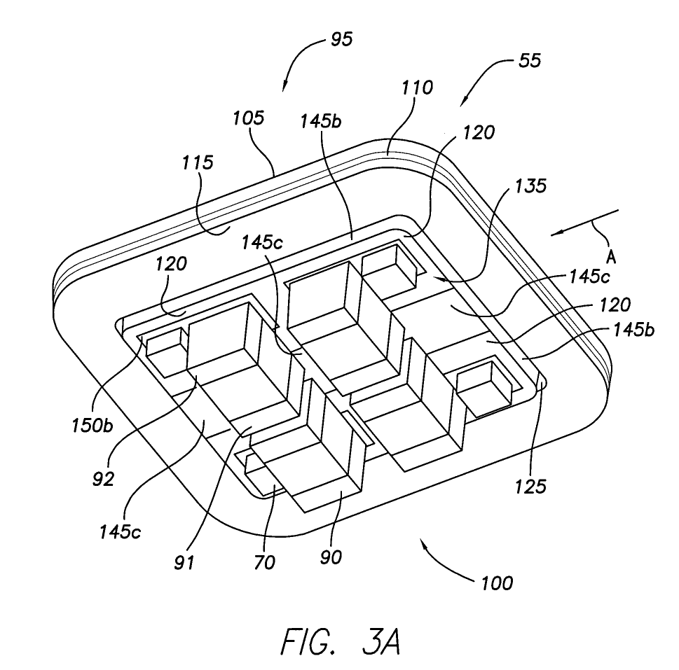

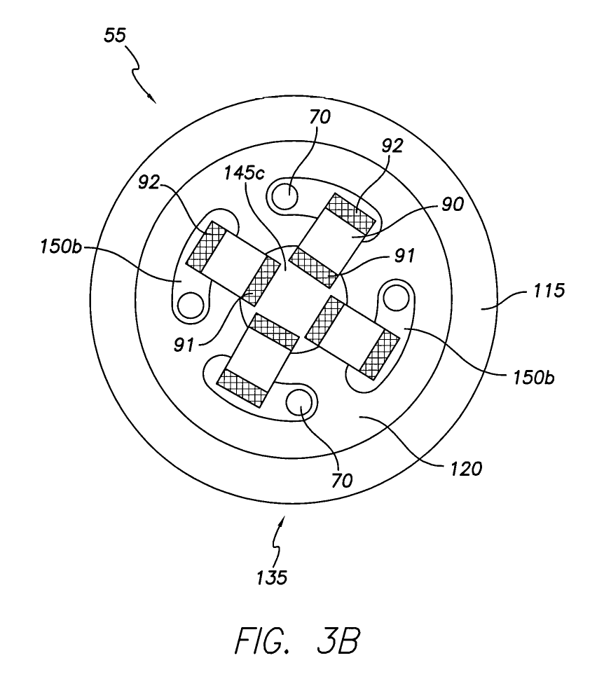

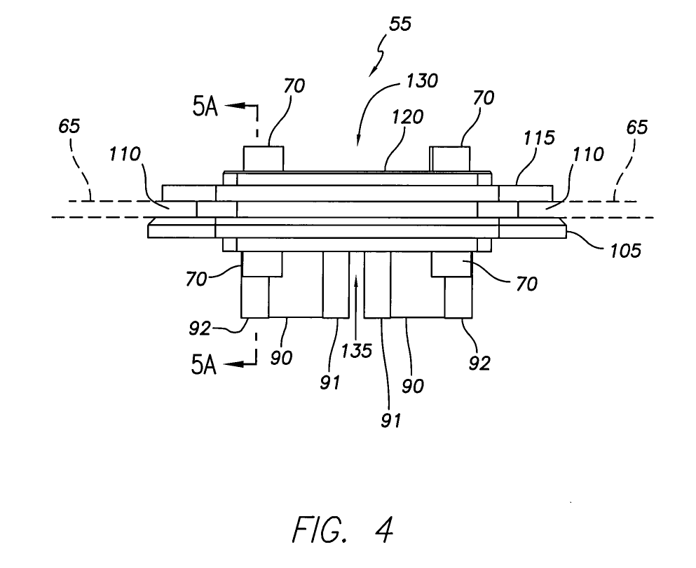

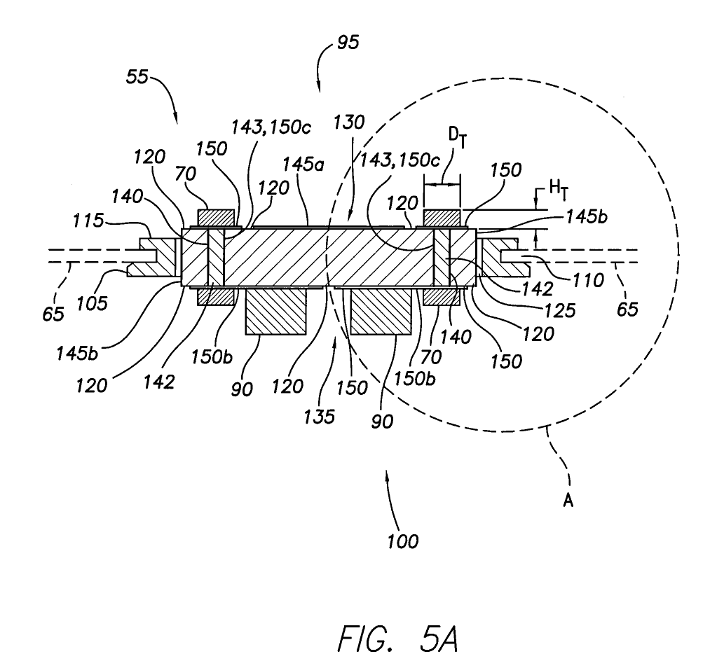

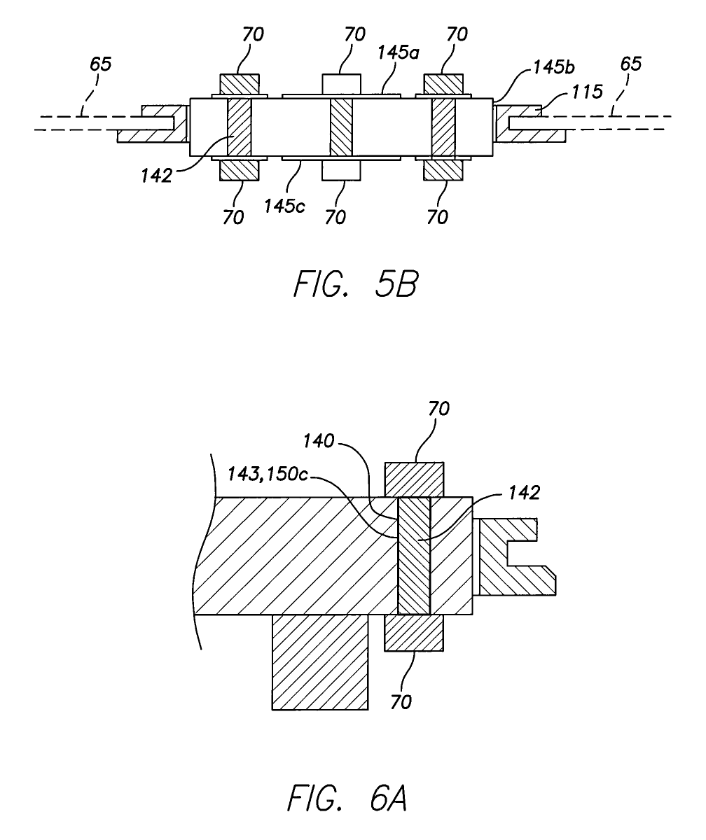

For a detailed discussion of the components of the feedthru 55, reference is now made to FIGS. 2A-5B. FIG. 2A and FIG. 3A are, respectively, top and bottom isometric views of the feedthru 55 of FIG. 1. FIG. 2B and FIG. 3B are, respectively, top and bottom plan views of an alternative embodiment of the feedthru 55 of FIG. 1. FIG. 4 is a side view of the feedthru 55 taken from the direction of arrow “A” of FIG. 3A. FIG. 5A is a cross-sectional elevation of the feedthru 55 as taken along section line 5A-5A of FIG. 4. FIG. 5B is a longitudinal cross-sectional elevation of an alternative embodiment of the feedthru 55 as taken along section line 5B-5B of FIG. 2B, wherein the chip capacitors 90 are not shown for clarity purposes.

In one embodiment, as shown in FIGS. 2A, 3A and 4, the feedthru 55 includes a header side 95, a can side 100 and a lateral or edge side 105 that forms a rectangular or square edge or boarder of the feedthru 55. As can be understood from FIGS. 2B, 3B and 5B, in an alternative embodiment, the edge side 105 may form a circular or rounded edge or boarder of the feedthru 55. As indicated in FIGS. 4 and 5A, the edge side 105 may vary in diameter to define a slot or groove 110 that receives the wall 65 of the can 15 when the feedthru 55 is assembled into the can 15 of the pulse generator 5.

As can be understood from FIGS. 2A-5B, the feedthru 55 includes a feedthru housing 115, a core 120, chip capacitors 90, tabs 70 and ground and power circuits. The housing 115 forms the edge side 105 of the feedthru 55 and includes a central or core-receiving opening 125. The housing 115 may be machined, molded or otherwise formed to fit the space and design constraints of an implantable pulse generator 5. The housing 115 may be titanium, a titanium alloy, MP35N, or stainless steel.

The outer edge or boundary of the housing 115 is defined by the edge side 105 and includes the groove or slot 110 that receives the can wall 65 when the feedthru is mounted in the can wall. The central opening 125 of the housing 115 extends axially through the housing and defines a void that is occupied by the core 120.

As shown in FIGS. 2A-5B, the core 120 includes a header face 130, a can face 135, and through-holes 140 extending axially therethrough. The core 120 may be formed of an electrically insulating material, such as ceramic, glass, or sapphire.

As can be understood from FIGS. 1-5B, the feedthru 55 includes a power circuit and a ground circuit. The power circuit includes the tabs 70, their respective vias 142 and power traces 150. The tabs 70 are electrically coupled to each other by their respective vias 142, and the power traces 150 electrically couple the tabs 70 to the power sides 92 of the chip capacitors 90 located on the feedthru 55. The power circuit, via the tabs 70, electrically couples the power sides of the electrical components 17 housed in the can wall 65 to the lead connector blocks 20 of the header 10.

The ground circuit includes the feedthru housing 115 and ground traces 145 electrically coupled to the feedthru housing 115. The ground traces 145 electrically couple the ground sides 91 of the chip capacitors 90 to the feedthru housing 115, which is electrically coupled to the can wall 65. A detailed discussion regarding each of the components of the power and ground circuits is given below.

As indicated in FIGS. 2A-5B, the electrically conductive tabs 70 may be located on one or both of the faces 130,135 of the core 120. For example, tabs 70 may be located near each of the four corners of each face 130,135. The tabs 70 may be arranged such that a tab 70 on the header face 130 near a first corner of the feedthru 55 is located directly across the core from a tab 70 mounted on the can face 130 near the same first corner, thereby forming a pair of tabs 130. Such a paired arrangement may be provided at each of the four corners of the feedthru 55.

While in some embodiments, as illustrated in FIGS. 2A-3B, the tabs 70 are located near outside edges of the core header face 130 and core can face 135, in alternative embodiments, the tabs 70 may be located closer to the centers of the core header face 130 and core can face 135. In still other embodiments, tabs 70 may be located near both the centers and the outside edges of the core header face 130 and core can face 135. In other embodiments, the tabs 70 may be located in other configurations or locations as long as there is sufficient space for connection of the conductors 60, 62 to the tabs 70.

As can be understood from FIGS. 2A-3B, the number of tabs 70 on the core header face 130 generally corresponds to the number of tabs 70 on the core can face 135. In one embodiment, there are four tabs 70 on the core header face 130 and a corresponding four tabs 70 on the core can face 135. In some embodiments, there are less than four tabs 70 or more than four tabs 70 on each of the core header face 130 and the core can face 135.

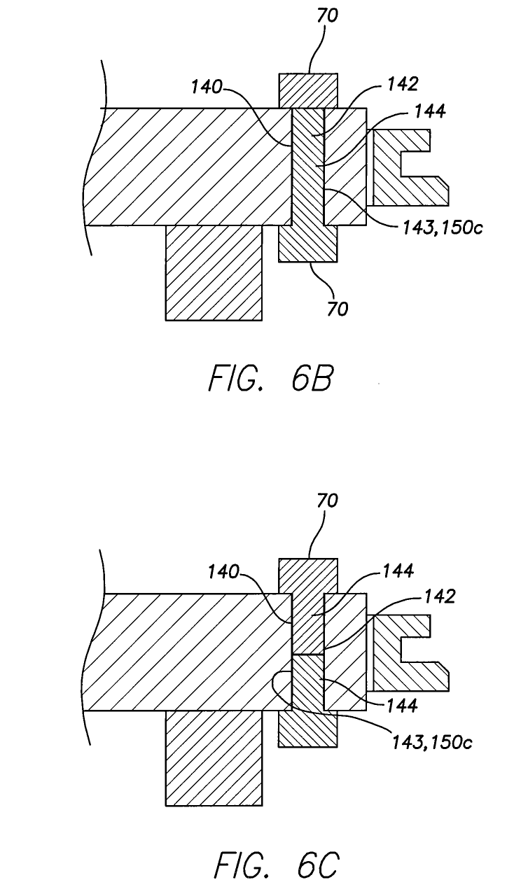

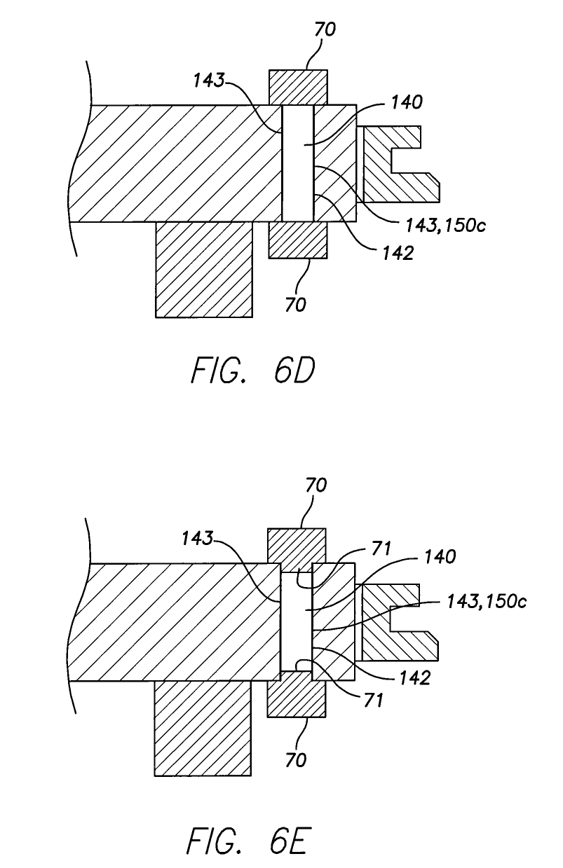

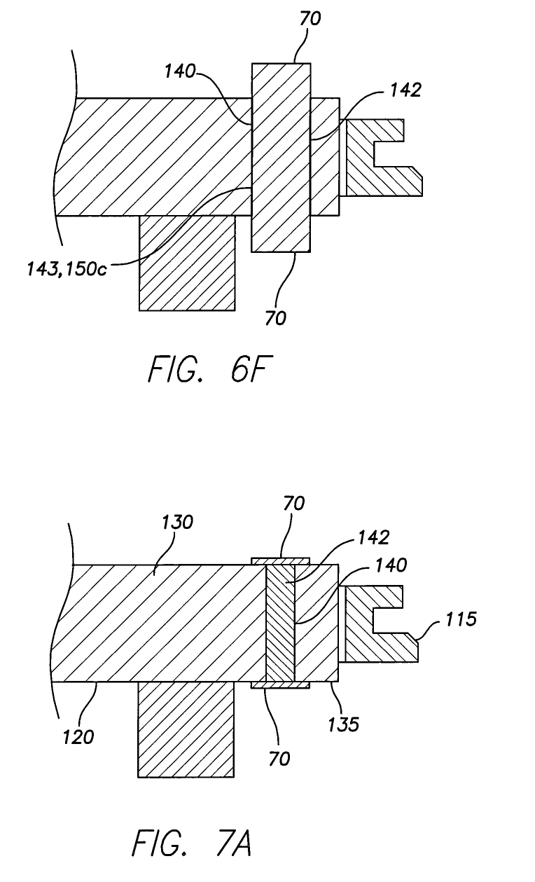

As indicated in FIGS. 5A and 5B, electrically conductive vias 142 extend through the through-holes 140 to electrically couple together the tabs 130 of each pair of tabs 70. The vias 142 and the associated tabs 70 may have a variety of configurations as shown in FIGS. 6A-6F, which are cross-sectional views of alternative tab configurations as if viewed in region A of FIG. 5A. For example, as indicated in FIG. 6A, the vias 142 may be a solid member 142 formed of electrically conductive material such as titanium, stainless steel, MP35N, etc. or a solid member formed of electrically or non-electrically conductive material coated with an electrically conductive material, such as gold, nickel, platinum, etc., where such coating is provided via electroplating, photo deposition, vapor deposition, etc. The surfaces of the through-holes 140 may additionally be coated with an electrically conductive material 143, such as gold, nickel, platinum, etc., where such coating is provided via electroplating, photo deposition, vapor deposition, etc. Such solid member vias 142 may be brazed (including gold brazed), welded or epoxied into the through-holes 140.

While the solid member vias 142 depicted in FIG. 6A may be a body that is a separate piece from the tabs 70 such that generally no portion of a tab 70 extends into a through-hole 140. As shown in FIGS. 6B and 6C, a portion 144 of a tab 70 may extend into the through-hole 140 to form at least a portion of a solid member via 142. For example, as depicted in FIG. 6B, the entirety of a solid member via 142 may be an extension 144 of a tab 70. Similarly, as illustrated in FIG. 6C, a portion of a solid member via 142 may be an extension 144 of both its respective tabs 70, each tab forming a portion of the solid member via 142. The tab 70 may also be a continuous, solid body extending all the way through the core 120 and also forming the solid member via 142. As shown in FIG. 6F, the diameter of the tab 70 may be the same as the diameter of the via 142. As can be understood from FIG. 6F, such a continuous, solid body tab 70 may be brazed to the through-hole 140 of the core 120. The solid body tab 70 may be made of titanium, MP35N, stainless steel, etc.

As shown in FIG. 6D, the vias 142 may be a hollow shaft extending axially through the core 120, wherein the surfaces of the hollow shafts are coated with an electrically conductive material 143 to form an electrically conductive trace or coating over on the surfaces of the hollow shafts. The vias 142 may be brazed, welded or secured to the tabs 70 via an electrically conductive epoxy.

As shown in FIG. 6E, in the vias 142 and tabs 70 may be a combination of the concepts shown in FIGS. 6B-6D, such that the tabs 70 partially extend into the through-holes 140 as nubs 71 and the through holes 140 with their electrically conductive coatings that serve as vias 142 complete the electrical connections between opposed nubs 71. Thus, the vias 142 could be described as a combination of electrically conductive coatings and nubs.

As can be understood from FIGS. 1 and 2A and 2B, conductors 60 leading to the connector blocks 20 are electrically connected via welding, brazing, etc. to the tabs 70 on the header face 130 of the core 120. In a similar fashion and as can be understood from FIGS. 1 and 3A and 3B, conductors 62 leading to the electrical components 17 (e.g. the output flex, hybrid, etc.) housed in the can 15 are electrically connected via welding, brazing, etc. to the tabs 70 on the can face 135 of the core 120. Thus, the tabs 70 and vias 142 provide an electrical pathway through the feedthru 55 to electrically couple the conductors 60, 62 and the connector blocks 20 and components 17 electrically coupled to the conductors 60, 62. As can be understood from FIGS. 1-5B, in at least some of the embodiments of the feedthrus 55 disclosed herein, the feedthrus 55 do not employ feedthru wires.

As can be understood from FIG. 5A, the tabs or posts 70 have a height H_{T} of between approximately 0.01 in. and approximately 0.05 in. and a diameter D_{T} of between approximately 0.03 in. and approximately 0.05 in. In one embodiment, the tabs or posts 70 have a height H_{T} of approximately 0.02 in. and a diameter D_{T} of approximately 0.03 in. The tabs 70 may be formed of titanium, kovar, stainless steel, MP35N, platinum or gold. The tabs 70 may be brazed, welded or secured to the core faces 130, 135 via an electrically conductive epoxy. In one embodiment, the via 142 may have a length that is generally the same as the thickness of the core 120, e.g., 0.06 in. In one embodiment, the via 142 may have a diameter of 0.015 in.

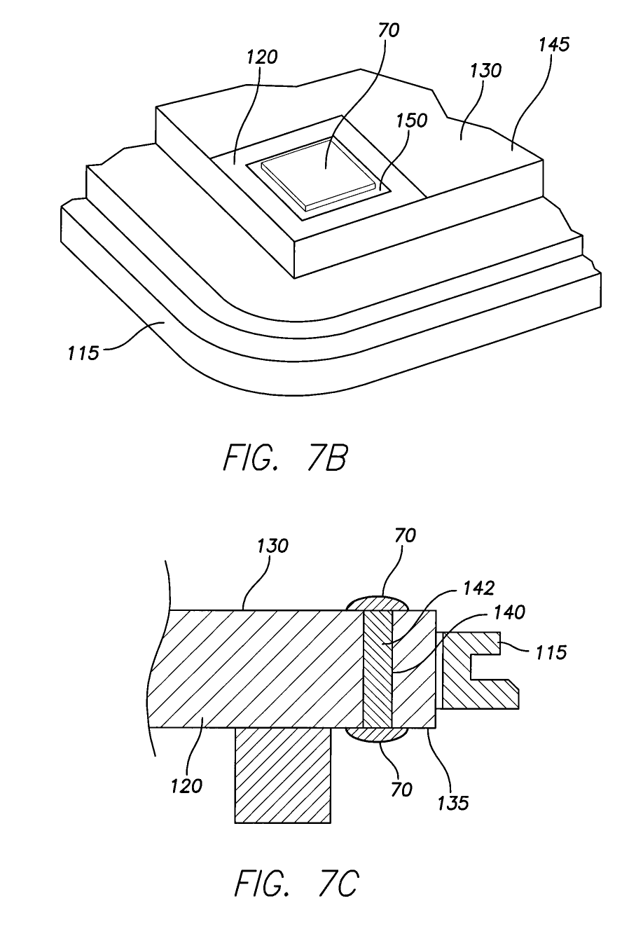

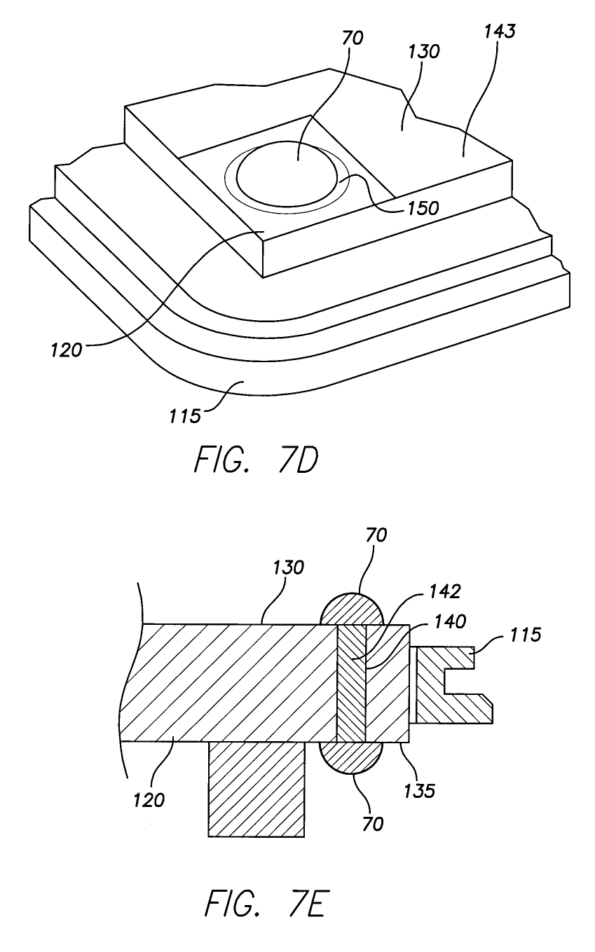

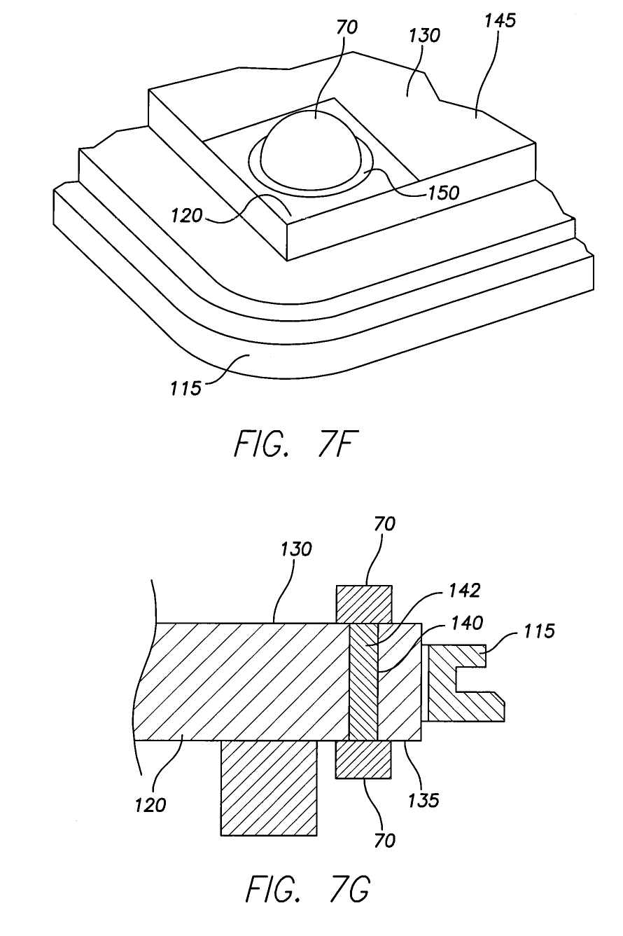

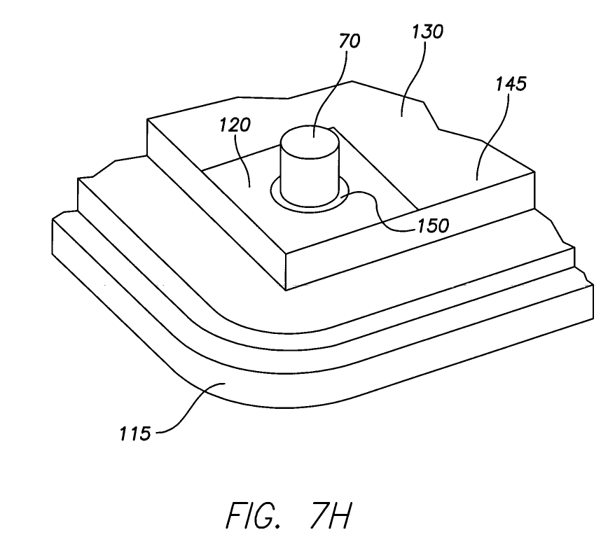

In some embodiments, as can be understood from FIGS. 7A-7D, which are cross sectional and isometric views, the tabs 70 may have a low surface relief. In some low-relief embodiments, as shown in FIGS. 7A-7B, the tabs 70 may appear flat and perhaps even nearly flush with the core header and can surfaces 130, 135 on which the tabs 70 are mounted. In some other low relief embodiments, as depicted in FIGS. 7A-7B, the tabs 70 may be slightly raised to be bump-like. In any of the embodiments depicted in FIGS. 7A-7D, the low relief tabs 70 may have a circular, rectangular or some other configuration. The low relief tabs 70, whether flush or bump-like, simply serve as locations or features for welding, brazing or other types of attachment to the conductors 60, 62 of the header 10 and can 15.

In some embodiments, as can be understood from FIGS. 2A and 5A and 5B, the tabs 70 may have a post-like configuration that projects a small distance from the core header and can surfaces 130, 135 on which the tabs 70 are mounted and, as a result, are less low-relief than the embodiments discussed with respect to FIGS. 7A-7D. As indicated in FIGS. 2A and 5A, the post-like tabs 70 may be box-like or cubical in shape.

As can be understood from FIGS. 7E-7H, which are cross sectional and isometric views, the post-like tabs 70 may have other shapes or configurations. For example, as shown in FIGS. 7E-7F, the post-like tabs 70 may be half spherical or another rounded shape. As shown in FIGS. 7G-7H, the post-like tabs 70 may have a cylindrical shape. In other embodiments, the post-like tabs 70 may have other shapes or configurations, such as cubical, half-spherical, cylindrical or some other shape. The post-type tabs 70, whether cubical, half-spherical, cylindrical or some other shape, simply serve as locations or features for welding, brazing or other types of attachment to the conductors 60, 62 of the header 10 and can 15.

While the tab configurations illustrated in FIGS. 2A and 2B, 5A and 5B and 7A-7H show matching tab configurations on each side of the core, in various embodiments, any one, two or more tab configurations depicted in FIGS. 2A and 2B, 5A and 5B and 7A-7H may be combined on a single feedthru 55 or paired with a single via 142. Similarly, while the via configuration depicted in FIGS. 5A and 5B show matching via configurations, any of one, two or more via 142 configurations depicted in FIGS. 6A-6F may be used on a single feedthru 55.

As can be understood from FIGS. 2A and 3A, the outer boarder or edge surface of the core 120 is rectangular and, in one embodiment, square. In an alternative embodiment, as shown in FIGS. 2B and 3B, the outer edge is round or circular. As shown in FIGS. 2A-3B, the outer edge surface of the core 120 projects a small amount past the housing 115 on both the header and can sides 95, 100 of the feedthru such that the outer edge surface of the core 120 is partially exposed and not entirely within housing 115.

As indicated in FIGS. 2A-3B, an electrically conductive ground coating or trace 145 extends over the core outer edge surface, a substantial portion of the core header face 130, and a smaller portion of the core can face 135. In one embodiment, the core header face 130 is generally entirely coated with the ground trace 145a, except in small regions 143 surrounding the tabs 70, wherein the small regions 143 are exposed surfaces of the core 120 electrically isolating the tabs 70 from the ground trace 145.

The ground trace 145b extends along the core outer edge from the core header face 130 to generally cover the entire surface of the core outer edge. The ground trace 145b extending over the core outer edge is in electrical contact with, and brazed or welded to, the housing 115, which is in electrical contact with the can wall 65. The can wall 65 serves as the ground for the pulse generator 5.

The ground trace 145c extends across the center of the core can face 135 from the core outer edge in the form of a rectangular trace 145c, in the context of FIG. 3A, and a round trace 145c, in the context of FIG. 3B. Chip capacitors 90 are located on the core can face 135. The ground trace 145 in all of its locations acts as a portion of the ground circuit, coupling the ground sides 91 of the chip capacitors 90 to the can wall 65 via the feedthru housing 115, which is another portion of the ground circuit. The can wall 65, which is electrically coupled to the feedthru housing 115, serves as the ground for the pulse generator 5. The ground trace 145 in any of its locations may be made of gold, platinum, nickel, titanium, or MP35N. The ground trace 145 in any of its locations may be formed via any method, including photo etching, deposition, electroplating, etc.

As shown in FIGS. 2A and 2B, an electrically conductive power coating or trace 150a boarders each tab 70 and is separated from the adjacent ground trace 145a, 145b by an exposed region 143 of the surface of the electrically insulating core 120. As indicated in FIGS. 3A and 3B, an electrically conductive power trace 150b extends across the core can face 135 from a tab 70 surrounded by the power trace 150b to a power side 92 of a chip capacitor 90. The power trace 150b may extend along the core can face 135 in the form of a rectangle or an oval or other suitable shape.

As indicated in FIGS. 5A-6D, power traces 150c, in the form of electrically conductive coatings 143, may extend along the vias 142 and/or the surfaces of the through-holes 140 to join with the power traces 150a, 150b on the core header and can sides 130, 135. The power traces 150a, 150b, 150c form a power side electrical circuit, along with the tabs 70 and vias 142, that electrically couples the power sides 92 of the chip capacitors 90 with the connector blocks 20 and electrical components 17 via the conductors 60, 62. The power traces 150 may be formed of any electrically conductive material (e.g. gold, platinum, nickel, titanium, MP35N, etc.) capable of being formed into a trace via any method including photo etching, deposition, electroplating, etc.

As can be understood from FIGS. 2A-3B and with reference to FIG. 1, the tabs 70 on the core header face 130 may be electrically connected to the connector blocks 20 by conductors 60, such as round wires, flat ribbon wires or flex cables. At the core can face 135, the tabs 70 may be electrically connected to the electrical components 17 by conductors 62, such as round wires, flat ribbon wires or flex cables or to electrically conductive traces on a printed circuit board. Because the tabs 70 may be electrically connected to each other by vias 142 and electrically connected to the header and can components 20,17 by less expensive conductors, expensive feedthru wires, such as Pt/Ir wires, are not required in embodiments of the feedthru 55. Therefore, such feedthrus 55 have reduced material and manufacturing costs.

As illustrated in FIG. 3A and 3B, the chip capacitors 90 on the core can face 135 of may include a ground end 91 and a power end 92. The ground end 91 of the chip capacitor 90 is electrically connected to the ground trace 145. The power end 92 of the chip capacitor 90 is electrically connected to the power trace 150. In one embodiment, a first chip capacitor 90 is separated from a second chip capacitor 90 by a minimum of approximately 0.03 in.

The chip capacitors 90 are easy to obtain, that is, they are readily commercially available or “off-the-shelf” chip capacitors. For example, the chip capacitors 90 may be obtained as model 0805 chip capacitor as manufactured by NovaCap of Valencia, Calif. 91355. The chip capacitors 90 are a part of the EMI filter element. EMI is a (usually undesirable) disturbance caused in a radio receiver or other electrical circuit by electromagnetic radiation emitted from an external source. Such a signal may interfere with the electrical components in the can of the implantable pulse generator. Thus, an EMI filter element, such as a chip capacitor, may reduce or eliminate the interference caused by an EMI. Additionally, an “off-the-shelf” chip capacitor may be less expensive and easier to obtain than a discoidal filter assembly, thus reducing the design and manufacturing costs of the feedthru 55.

As can be understood from FIGS. 4 and 5A and 5B, to assemble the feedthru 55, the housing 115 and core 120 may be connected by soldering, brazing, welding or other suitable method to form a housing-core assembly. The coupling of the core 120 to the housing 115 creates a hermetic seal. The tabs 70 may be connected to the core 120 by brazing, soldering, welding or other suitable method. The chip capacitors 90 may be surface mounted or otherwise connected to the can end 135 of the core 120 by soldering, electrically conductive epoxy or other suitable method.

As can be understood from FIGS. 2A-3B, and with reference to FIG. 1, the feedthru 55 is assembled into the can wall 65 and electrically coupled to the electronic components 17 in the can 15 and the lead connector blocks 20 in the header 10. The can wall 65, which is electrically coupled to the feedthru housing 115, is in electrical communication with the ground side 91 of the chip capacitor 90 via the ground circuit extending through the feedthru housing and ground trace 145. Similarly, to the electronic components 17 in the can 15 and the lead connector blocks 20 in the header 10 are in electrical communication with each other and the power side 92 of the chip capacitor 90 via the power circuit formed by the tabs 70, their respective vias 142 and the power trace 150.

Although the present invention has been described with reference to preferred embodiments, persons skilled in the art will recognize that changes may be made in form and detail without departing from the spirit and scope of the invention.

What is claimed is:

1. An implantable pulse generator comprising:
a header including a lead connector block electrically coupled to a first conductor;
a can coupled to the header and including a wall and an electronic component electrically coupled to a second conductor and housed within the wall; and
a feedthru mounted in the wall and including a header side with a first electrically conductive tab and a can side with a second electrically conductive tab electrically coupled to the first tab, wherein the first tab is electrically coupled to the first conductor and the second tab is electrically coupled to the second conductor.

2. The pulse generator of claim 1, further comprising a chip capacitor on a can side of the feedthru.

3. The pulse generator of claim 2, wherein the feedthru includes an electrically conductive ground trace electrically coupled to the wall and a ground side of the chip capacitor.

4. The pulse generator of claim 3, further comprising an electrically conductive power trace electrically coupled to the tabs and a power side of the chip capacitor.

5. The pulse generator of claim 3, wherein the ground trace extends over surfaces of the header side and can side.

6. The pulse generator of claim 1, wherein at least one of the tabs is post-like.

7. The pulse generator of claim 6, wherein the post-like tab is generally cubical, generally cylindrical or generally half-spherical.

8. The pulse generator of claim 1, wherein at least one of the tabs is of a low relief relative a surface of the side on which the at least one of the tabs is mounted.

9. The pulse generator of claim 8, where the at least one of the tabs is of such low relief as to be at least nearly flush with the surface.

10. The pulse generator of claim 1, further comprising an electrically insulating core including between the first tab and the second tab.

11. The pulse generator of claim 10, wherein the core includes a through-hole through which an electrically conductive path extends between the tabs.

12. The pulse generator of claim 11, wherein a surface of the through-hole is coated with an electrically conductive material, and the coating electrically couples the tabs to each other.

13. The pulse generator of claim 11, further comprising an electrically conductive member extending through the through hole and electrically coupling the tabs to each other.

14. The pulse generator of claim 13, wherein the member forms an extended portion of one of the tabs.

15. The pulse generator of claim 10, wherein the core is formed of a ceramic material.

16. The pulse generator of claim 1, wherein at least one of the conductors is at least one of round wire, flat ribbon wire, and flex cables

17. An implantable pulse generator feedthru comprising:
an electrically insulating body including a header side and a can side;
a ground circuit at least a portion of which is on the body; and
a power circuit including a first tab on one of the sides.

18. The feedthru of claim 17, wherein the tab has a low-relief configuration.

19. The feedthru of claim 18, wherein the low-relief configuration is generally flush with a surrounding surface on which the tab is located.

20. The feedthru of claim 18, wherein the low-relief configuration is bump-like.

21. The feedthru of claim 17, wherein the tab is has a post-like configuration.

22. The feedthru of claim 21, wherein the post-like configuration is cubical, cylindrical or half-spherical.

23. The feedthru of claim 17, further comprising a second tab, an electrically conductive via and a through-hole extending through the body, wherein the first tab is located on an end of the through-hole on the header side of the body, the second tab is located on an end of the through-hole on the can side of the body, and the via extends through the through-hole and electrically couples together the tabs.

24. The feedthru of claim 23, wherein the via is the through-hole coated with an electrically conductive coating.

25. The feedthru of claim 23, wherein the via includes: an empty portion of the through-hole coated with an electrically conductive coating; and a nub portion of at least one of the tabs extending into the through-hole.

26. The feedthru of claim 23, wherein the via includes an extension of one of the tabs extending through the through-hole to the other tab.

26. The feedthru of claim 23, wherein the via includes an extension of both of the tabs extending into the through-hole to meet each other.

26. The feedthru of claim 23, wherein the via includes a member separate from the tabs and extending through the through-hole from one tab to the other.

27. The feedthru of claim 17, further comprising a chip capacitor coupled to the body and including a power side electrically coupled to the power circuit and a ground side electrically coupled to the ground circuit.
